(19) United States
(12) Patent Application Publication
(10) Pub. No.: US 2025/0057938 A1
(43) Pub. Date: Feb. 20, 2025

(54) STABILISED LIQUID VACCINES OF LIVE VIRUSES

(71) Applicant: INTERVET INC., Madison, NJ (US)

(72) Inventors: Edwin KETS, Heilig Landstichting (NL); Martin PIEST, Afferden (NL); Paul VERMEIJ, St. Anthonis (NL)

(73) Assignee: INTERVET INC., Madison, NJ (US)

(21) Appl. No.: 18/723,259

(22) PCT Filed: Dec. 22, 2022
(86) PCT No.: PCT/EP2022/087464

§ 371 Date: Jun. 21, 2024

(30) Foreign Application Priority Data
Dec. 23, 2021 (EP) 21217481.7

(51) Int. Cl. A61K 39/155; A61K 39/00; A61K 39/175; A61K 39/215; A61K 39/225; A61K 47/18; A61K 47/20; A61K 47/22; A61K 47/26

(52) U.S. Cl. CPC A61K 39/155; A61K 39/175; A61K 39/215; A61K 39/225; A61K 47/183; A61K 47/20; A61K 47/22; A61K 47/26; A61K 2039/5254

(57) ABSTRACT

The present invention relates to liquid vaccine composition comprising a live virus and a natural deep-eutectic solvent (NADES) as the carrier. The carrier additionally comprises an additive selected from methionine and (hydroxy) ectoine. The additive is able to reduce the loss of virus titre over time, upon storage in a NADES-based liquid vaccine composition having up to 50% w/w of water. Such compositions are less viscous which is favourable for the manufacture of the carrier, and the formulation and use of the liquid vaccine.

### FIELD OF THE INVENTION

The present invention relates to the field of vaccinology, and specifically to vaccines of live viruses. In particular the invention relates to a liquid vaccine composition comprising a live virus and a natural deep-eutectic solvent (NADES) as the carrier. Further to methods for preparing the liquid vaccine composition, to methods for stabilising a live virus in such liquid compositions, and to medical uses of such liquid vaccine compositions.

### BACKGROUND OF THE INVENTION

Deep eutectic solvents (DES) are liquid compositions that combine a number of remarkable properties, which derive from the fact that such a composition is ionic but has very little free water. Essentially these are mixtures of two or more salts, whereby the melting point of the mixture is much reduced as compared to that of the individual components. The result is that such a mixture, even though it consists mainly of salts, is nevertheless liquid at ambient temperatures, or even below zero ° C.

DES were for example described by Abbott et al., 2003, Chem. Commun., vol. 1, p. 70-71, and in WO 2009/120839. Smith et al. (2014, Chem. Rev., vol. 114, p. 11060-11082) have proposed a categorisation of DES into 4 classes. A recent review on the properties and microstructure of DES is Kaur et al., 2020 (J. of Phys. Chem. B, vol. 124, p. 10601-10616).

As ionic liquids, DES are very effective as solvents for a variety of chemical- and industrial processes. Examples range from the extraction of gasses, minerals, and industrial bulk products, to solvents for metals and pharmaceuticals. DES have also been used for the extraction of biological molecules such as enzymes and RNA, see U.S. Pat. No. 8,247,198 and WO 2011/155829, respectively.

A special form of DES are those of which the components are naturally occurring substances. Such so-called ‘natural DES’, or NADES, were described in: Choi et al. 2011 (Plant Physiol., vol. 156, p. 1701-1705); and: Dai et al. 2013 (Anal. Chim. Acta, vol. 766, p. 61-68). NADES are currently being used as ‘green’ solvents for a variety of biological compounds such as cellulose, and vanillin.

As is well-known in this field, the critical features determining the immunological efficacy of a live viral vaccine are the amount and the characteristics of the virus used. For a given vaccine strain, the preservation of the minimal effective live virus titre (i.e. the potency) up until expiration of the registered shelf life period, is therefore of the utmost importance. The classic way of preserving live virus is by freeze-drying, however that is a laborious, energy-intensive, and expensive process.

In WO 2019/122329 liquid vaccine compositions were disclosed that overcome the need for freeze-drying, and that enabled the development of liquid vaccines of sensitive live enveloped viruses with excellent stability, even at elevated temperatures. This was achieved by the use of a NADES as the vaccine carrier and additive for the virus, because even the small amount of water that is present, is not readily available for chemical- or biological processes. In the '329 patent-application the NADES is preferably composed of an organic salt and a polyol, and the vaccines typically comprise less than 20% w/w water.

Several publications describe the stabilisation of virus in liquid vaccine compositions, and employ compounds that at first sight resemble those as described in '329, for example: WO 2011/121306, WO 2014/029702, WO 2014/140239, WO 2015/121463 and US 2014/0271710. However those liquid vaccines are not based on NADES, but on regular aqueous compositions. Such aqueous compositions differ essentially from NADES-based compositions in their physical properties and ability to stabilise sensitive viruses. In Example 1 hereinafter, several of these published aqueous compositions are analysed with regard to the concentration of their salts, their water amount, and their water activity. This immediately clarifies that those compositions are aqueous compositions and employ water as the pharmaceutically acceptable carrier, but are not NADES-based.

WO 2020/201048 describes NADES-based liquid vaccines of Mollicutes bacteria.

A DES consists of a hydrogen bond network of intermolecular interactions, in which the water that is present in that composition is tightly bound. This also makes that there is a maximal amount of water that can be contained by that DES network. The consensus in the art is that an amount of water up to about 50% w/w can be comprised in a DES before its network structure is fully disturbed, and the composition changes from being a DES to an aqueous composition. See e.g.: Dai et al., 2015 (Food Chem., vol. 187, p. 14-19); Hammond et al., 2017 (Angew. Chem., Int. Ed., vol. 56, p. 9782-9785); and Liu et al., 2018 (J. of Nat. Prod., vol. 81, p. 679-690).

While the NADES-based liquid vaccines of WO 2019/122329 could be administered by injection, some of those compositions can be quite viscous. This can be a disadvantage for their preparation and processing at large scale, and for certain methods of administration of such a vaccine.

It is therefore an object of the present invention to overcome a disadvantage in the prior art, and to accommodate to a need in the field by providing a liquid vaccine of live viruses based on a NADES as the pharmaceutically acceptable carrier, which vaccine has a reduced viscosity and can accommodate a relatively large amount of water, while reducing the loss in the viral titre.

### DESCRIPTION OF THE INVENTION

Surprisingly it was found that this object can be met, and consequently one or more disadvantages of the prior art can be overcome, by providing a NADES-based liquid vaccine of live viruses, which additionally comprises methionine, ectoine, or hydroxy-ectoine.

This discovery enables the development of NADES-based liquid vaccine compositions which have a reduced viscosity e.g. as compared to the compositions described in WO 2019/122329, and can contain an amount of water up to 50% w/w, but still provide good stabilisation of live viruses. These liquid vaccines are now easier to manufacture and formulate, especially at large scale, and allow easier handling and administration by different routes. Also, the relatively large amount of water, provides more flexibility on the inclusion of viruses or other antigens. For instance, this enables the inclusion of live virus from less concentrated aqueous preparations, and/or the additional inclusion of several other antigens, to compose multi-antigen combination vaccines.

Importantly, these vaccines still provide the advantages of NADES-based liquid vaccines, in that they provide excellent stabilisation of live virus even at temperatures above zero ° C., so do not require freeze-drying for their manufacture, nor require reconstitution before administration to a target, and can conveniently be stored at temperatures below zero ° C. without freezing over.

In their attempts to prepare less viscous NADES-based vaccines, the inventors quickly learned that merely increasing the amount of water was only a partial solution: while the viscosity was indeed lower, unfortunately the stabilising capacity was reduced too. Already when water amounts were increased over 5% w/w a reduction of viral titre was clearly noticeable in stability assays; this increased to modest to severe loss of live virus titre upon the inclusion of 20-40% w/w water respectively; and above 40% w/w very rapid loss of live virus titre was observed. When water was included to more than 50% w/w, the compositions lost their NADES properties, as displayed by a change in their differential scanning calorimetry (DSC) profile, from a glass-transition-to an ice crystallisation profile that is characteristic for an aqueous solution, as described herein.

The inventors then had to find a way to prolong virus survival in these NADES-based vaccines with reduced viscosity. One option considered was the addition of a further ingredient. However many different compounds were tested, but most did not provide good reduction of loss of virus titre. In fact only very few compounds, namely: methionine and (hydroxy) ectoine, were actually able to reduce the loss of virus titre over time, when live virus was stored in a NADES-based liquid vaccine composition according to the invention comprising up to 50% w/w of water.

This was unexpected, and it clearly showed that any virus-stabilising properties of compounds in regular aqueous compositions, are not likely to occur also in conditions that are physically very different such as in a NADES-based composition with very low levels of free water. Consequently preceding publications of stabilising effects by compounds used in aqueous compositions, cannot be used to predict, generalise, or extrapolate such effects to occur also in the context of a NADES-based composition.

It is not known exactly how or why methionine and (hydroxy) ectoine are able to prolong the viability of live virus in NADES-based liquid vaccine compositions having up to 50% w/w of water. This was particularly surprising as several of the compounds used to form a NADES are themselves considered to have stabilising and/or antioxidative effects, and those compounds are present in large excess compared to the added methionine or (hydroxy) ectoine.

Although the inventors do not want to be bound by any theory or model that might explain these findings, they speculate that methionine, ectoine, and hydroxyectoine perhaps display this favourable effect because they can in some way or other avert the negative effect of the water that is present, on the titre of the live virus over time.

Therefore in one aspect the invention relates to a liquid vaccine composition comprising a live virus and a pharmaceutically acceptable carrier, the carrier is a natural deep-eutectic solvent (NADES), and the vaccine has a water activity of at most 0.8, characterised in that the liquid vaccine composition has a water amount of up to 50% w/w, and the vaccine also comprises an additive selected from methionine, ectoine, and hydroxyectoine.

The term “liquid” is used in its common meaning, referring to a composition that can flow at a certain temperature and within a certain time period. To determine liquid properties a tilt test is performed wherein a container with a composition at a certain temperature (e.g. ambient temperature) is tilted, and when after a certain amount of time (e.g. 30 minutes) a change of the form of the composition is detected, that composition is classified as being a liquid.

A “vaccine” is well known to be a composition that has a medical effect. A vaccine comprises an immunologically active component, and a pharmaceutically acceptable carrier. The ‘immunologically active component’, in the present case comprises a live virus. After administration of the vaccine the immunogen is recognised by the immune system of the target human or animal, which induces a protective immunological response. The response may originate from the targets' innate- and/or from the acquired immune system, and may be of the cellular- and/or of the humoral type.

A vaccine generally is efficacious in reducing the severity of an infection, for example by reducing the number of the pathogens, or shortening the duration of the pathogen's replication in a host.

Also, or possibly as a results thereof, a vaccine generally is effective in reducing or ameliorating the (clinical) symptoms of disease that may be caused by such infection or replication of a pathogen, or by the target's response to that infection or replication.

The term “comprising” (as well as variations such as “comprise”, “comprises”, and “comprised”) as used herein, intends to refer to all elements, and in any possible combination conceivable for the invention, that are covered by or included in the text section, paragraph, claim, etc., in which this term is used, even if such elements or combinations are not explicitly recited; and not to the exclusion of any of such element(s) or combinations.

Thus any such text section, paragraph, claim, etc., can therefore also relate to one or more embodiment(s) wherein the term “comprising” (or its variants) is replaced by terms such as “consist of”, “consisting of”, or “consist essentially of”.

While strictly speaking it is not biologically correct to refer to a virus as being “live”, that is the common way to refer to a virus that is not inactivated. Consequently, for the invention the term “live” refers to a virus that is capable of replication (i.e. is ‘replicative’) under appropriate conditions, e.g. in suitable host cells.

In practice the live virus comprised in the liquid vaccine composition according to the invention will be a virus that is suitable for vaccination of a human or animal target. Typically this means the vaccine virus has a reduced pathogenicity for that target. This phenotype can result from the natural properties of the virus itself, e.g. of being less pathogenic when used in a particular target species.

Alternatively the virus can be a virus that has been attenuated; such a virus is also called ‘modified live’. “Attenuated” for the invention is defined as causing a lower level of disease symptoms, e.g. by having a reduced rate of infection or of replication, as compared to the unmodified, non-attenuated, or ‘wildtype’ form of such a virus. Attenuation of a virus can be obtained in different ways, for example by passageing and selection through experimental animals or in cell-culture, or by random- or targeted mutation, e.g. via chemicals, irradiation, or via recombinant DNA technology; all well known in the art.

A virus is a well-known micro-organism, of which many types can be pathogenic to humans or animals. A wide variety of virus types and—families has been described in handbooks such as Fields Virology (4th Edition 2001, Lippincott Williams & Wilkins, ISBN-10:0781718325).

For the invention, the ‘titre’ of a composition comprising live virus refers to the amount of infectious virus in that composition. A reduction of that titre results from virus losing its capacity to infect host cells, and/or losing it capacity to replicate once inside a host cell. This may be caused by damage to e.g. the viral envelope, structural proteins, and/or nucleic acid. Consequently, the stability of a live virus can effectively be indicated in terms of the effect on its titre. This can be determined in vivo, but more conveniently in vitro, for example using fertilised eggs or a cell-culture of suitable host cells. Viral titre can then e.g. be compared before and after storage, and be expressed e.g. in terms of the tissue culture infective dose (TCID50), cell infectious dose (CID), plaque forming units (pfu), or egg infective dose (EID).

For the invention, a “pharmaceutically acceptable carrier” is a liquid of a high grade of purity and suitable for medical purposes. In the present case the carrier is a NADES. The carrier can comprise further excipients.

A “deep-eutectic solvent” (DES) is well-known in the art as an ionic liquid comprising a mixture of at least two compounds at a molar ratio that forms a eutectic mixture such that the eutectic point of the resulting mixture is significantly lower than the melting points of the individual compounds. This reduced melting point of the mixture is caused by the molecular interactions of the compounds, one acting as proton donor, and one other acting as proton acceptor. This allows the generation of stable hydrogen-bonding without crystallisation, allowing the mixture to be in liquid form at much reduced temperatures, as compared to its constituents. Commonly ‘eutectic’ means: easy melting.

For the invention, the individual compounds used to form a DES for the invention have melting points above about 80° C., and the DES has a melting point below about 40° C. For example, the melting point temperatures of proline and of sorbitol are 228° C. and 112° C., respectively, while a NADES formed at a molar ratio for proline, sorbitol, and water of 1:1:2.5, formed a clear liquid at ambient temperatures, and even remained fluid at −20° C.

A DES thus mainly consists of liquified salts, optionally with a small amount of water. A DES is therefore essentially different from an aqueous composition, as that consists mainly of water with an amount of salt in it. This difference between these two types of liquids is also clear from comparing parameters such as water activity (a_{w}) and water amount (in % w/w), and is also reflected by the difference in their DSC profiles; the DSC profiles of aqueous compositions show the forming and melting of ice crystals, but a NADES has a glass-transition profile. Details are described herein.

For the invention “about” indicates that a number can vary between ±10% around its indicated value. Preferably, about means±9% around its value, more preferably about means±8, 7, 6, 5, 4, 3, 2% around its value, or even about means±1% around its value, in that order of preference.

In the terms “natural deep-eutectic solvent” and “NADES”, the feature “natural” serves to indicate that the compounds used to form this type of DES, are organic compounds that are present in material from biological sources such as plants or animals, in amounts well above trace amounts. Typically such a natural compound is, or is derived from, a primary metabolite that is present in a specific material of vegetable or animal origin. As the skilled person will appreciate, the term natural is only used herein to characterise the initial origin of a compound, and not to characterise the way the compound being used in a NADES for the invention was actually sourced. Thus the natural compound for the invention can be obtained from (semi-) synthetic production.

Examples of natural compounds that can be used to form a NADES, are organic acids, amines, sugars, sugar alcohols, and amino acids.

A further advantageous characteristic of the liquid vaccine composition according to the invention, is that the water that is present in the vaccine is tightly bound in the structure of the NADES. The result of this is that the amount of water that is available for chemical or biological processes, e.g. to influence the stability of the live virus, is very limited. This feature is commonly expressed by the value of the “water activity”, indicated by the symbol: a_{w}. A water activity can vary between the upper limit of 1.0 for pure water, and the lower limit of 0. Water activity is commonly measured by comparing (at the same temperature) the vapour pressure of a test composition, relative to that of pure water and to a number of saturated salt-solutions of known water activity. This is described in different handbooks, reviews and manuals, such as for example on the conservation of fruits and vegetables in the FAO agricultural service bulletin no. 149 (Cánovas et al, FAO, Rome, 2003, ISBN 92-5-104861-4); and a review is in ‘Fundamentals of water activity’, Decagon Devices Inc., Washington, 2015 (http://pdf.directindustry.com/pdf/decagon-devices-inc/fundamentals-water-activity/64142-634433.html).

At a water activity of 0.8 or less the growth of most bacteria is stopped; from 0.7 growth of most yeasts and moulds is stopped, and at a water activity below 0.4 most enzyme activity is effectively stopped.

Equipment and procedures to measure water activity are well known and available, for instance by using headspace pressure analysis.

For the invention, the indicated water activity refers to the water activity of the liquid vaccine composition according to the invention in the form of a final product, for example such as is offered by a commercial producer, and in which form it can be stored for prolonged time, before its administration to a target. The indicated water activity thus does not relate e.g. to dilutions of the liquid vaccine compositions according to the invention made shortly before administration to a target, e.g. upon mixing with another vaccine, or when making dilutions for administration e.g. by spray or via the drinking water.

The liquid vaccine according to the invention contains other ingredients aside from the NADES, and these other ingredients may comprise water, e.g. an aqueous stock solution of live virus. Consequently, the water activity of the NADES carrier will typically be less than that of the final liquid vaccine composition, in order to allow for the addition of such other ingredients, and not exceed the maximal water activity of the liquid vaccine for the invention.

For the invention, the “water amount” indicates the amount of water present in the composition of the liquid vaccine according to the invention. It is calculated by dividing the weight of the total amount of water in the liquid vaccine, by the total weight of that vaccine, and expressed as a percentage.

Water amount can be measured using a variety of different procedures, for example by Karl Fischer titration, well known in the art.

In the experiments leading up to the present invention, samples of NADES-based vaccines with live virus were prepared and stored for increasing duration at a specific temperature e.g. at −20, 4, 20 or 37° C. Subsequently the remaining viral titre after storage was determined. As disclosed in detail in the Examples hereinafter, for increasing water amounts the additive as described for the invention could reduce the loss of virus titre over time, when compared to a similar composition but without that additive. Therefore an additive as defined for the invention can reduce the loss of titre of a live virus that is comprised and stored in NADES-based compositions comprising up to 50% w/w of water.

Evidently, the advantages of a liquid vaccine according to the invention are lost when the composition is so much diluted that it loses its NADES properties and becomes an aqueous composition, i.e. from a water amount of about 50% w/w. As the skilled person will appreciate, the precise border for loss of NADES integrity is dependent on the particulars of the composition considered. Therefore the water amount of the liquid vaccine according to the invention is limited on the high side to a water amount up to 50% w/w.

Like for the water activity, the water amount of the liquid vaccine according to the invention, regards the liquid vaccine in the form of a final product, before any dilution is applied e.g. shortly before administration to a target.

By limiting the amount of water to 50% w/w or less, the use of a NADES as the carrier for the liquid vaccine composition according to the invention, provides said vaccine with an intact hydrogen-bond network typical for a DES. This can conveniently be verified by thermal analysis of the vaccine's phase-transition profile using differential scanning calorimetry (DSC). Consequently, a liquid vaccine according to the invention has a DSC profile that shows a glass-transition temperature, and not an ice crystallisation profile.

The determination of a DSC profile is well known and can be done using standard equipment. An example of the glass-transition profile of an intact NADES is presented in FIG. 1 of WO 2020/201048; represented herein as FIG. 1. Specifically this is the DSC profile of a NADES with proline, sorbitol, and water at 1:1:2.5 molar ratios.

Examples of DSC profiles that display the melt- and a crystallisation enthalpy of aqueous compositions that resulted from the over-dilution of NADES, are as presented in FIG. 4 of Qiao et al., (2018, Appl. Microbiol. Biotechnol., vol. 102, p. 5695-5705); represented herein as FIG. 2.

The term “additive” is used for the invention in the common meaning of this term, and refers to an added compound, e.g. an excipient. The additive for the invention can reduce the loss of infectivity of a live virus, in a liquid vaccine composition according to the invention, e.g. upon storage.

The results in the Examples section show that some loss of titre can still occur in the compositions of the invention. However, and as the skilled person will appreciate, even a reduction in the rate of loss of titre as compared to a control, signifies a valuable effect in virus stabilisation. Mainly as it can overcome a need for freeze-drying. In addition, it may e.g. allow to assign a 2 year shelf-life to a liquid vaccine product, which would not be possible without the present invention. So although clearly desirable, the criterion for an advantageous effect is not: zero loss of titre, but: a meaningful reduction to the rate or to the total level of titre loss, when compared to a similar composition that does not contain the additive of the invention, or is not NADES-based.

‘Methionine’ is a well-known amino acid; ‘ectoine’ and its derivative: ‘hydroxyectoine’, are well known compounds of bacterial origin.

Details of embodiments and of further aspects of the invention will be described below.

For the invention, the favourable effect of the presence of the additive in a liquid vaccine composition according to the invention manifests itself by a reduction of the loss of virus titre, as compared to the titre remaining after a control incubation. The control incubation is preferably done with a similar composition, which is preferably kept under similar conditions of storage such as temperature and duration. Preferably the reduction to the loss of titre provided by the presence of the additive is at least 5% after incubation during a certain period; this means that the virus titre in the composition with the additive preferably is at least 5% higher as in a control incubation composition after the incubation period.

The duration of the incubation (storage) period is preferably at least 1 week at 20° C., or at least 1 month at 4° C. More preferably at least 2, 3, 4, 6, 12, 18 or even 24 months at 4° C.

Preferably the reduction in the loss of virus titre provided by the presence of the additive is at least 10%, 15%, 20%, 25%, 30%, 35%, 40%, 45%, 50%, or even at least 75%, in this order of preference.

In an embodiment of the liquid vaccine composition according to the invention, the NADES is formed from an organic salt, a polyol, and water. The organic salt is preferably one or more selected from salts of: betaine, proline, carnitine, and choline. The polyol is preferably a sugar or a sugar-alcohol; the sugar is preferably one or more selected from: fructose, maltose, sucrose, glucose and trehalose; the sugar-alcohol is preferably one or more selected from: glycerol, xylitol, mannitol, and sorbitol.

In a preferred embodiment of the liquid vaccine composition according to the invention, the NADES is formed from proline, sorbitol and water.

‘Proline’ is a well-known amino acid; ‘sorbitol’ is a well-known sugar-alcohol.

In a preferred embodiment proline is CAS nr. 609-36-9.

In a preferred embodiment sorbitol is D-Sorbitol; more preferably D-Sorbitol is CAS nr. 50-70-4.

In a preferred embodiment the proline, sorbitol and water are present in the NADES in molar ratios of proline:sorbitol:water that are between 1:1:1 and 1:1:16.

For a NADES with a molar ratio of proline, sorbitol and water of 1:1:16, the amount of water is 49.2% w/w, and water concentration is at 32.3 M. However, as that leaves little room for including an aqueous virus preparation while staying below the 50% w/w amount of water in the vaccine, the molar ratio of proline:sorbitol:water in the NADES is preferably between 1:1:1 and 1:1:15, more preferably between 1:1:1 and 1:1:12, or even between 1:1:1 and 1:1:10.

Especially sensitive and therefore in need of proper stabilisation when in a liquid preparation, are live viruses that are relatively large, have an RNA genome, and/or have a viral envelope (i.e. are enveloped). Most sensitive are live viruses that are large, have an RNA genome, and have a viral envelope. This because they are sensitive to both physical- and chemical effects on their infectivity and replicative potential.

Therefore in an embodiment of the liquid vaccine composition according to the invention, the live virus is large, i.e. has a diameter of more than 50 nanometres. More preferably a large virus for the invention has a diameter of more than 75, 100, 150, or even more than 200 nanometres, in this order of preference.

In an embodiment of the liquid vaccine composition according to the invention, the live virus has an RNA genome.

The RNA genome can be in positive- or negative translational orientation, also known as: plus- or minus strand, or as: sense or antisense, respectively. As is well-known, the positive-, plus- or sense orientation allows for direct translation. The RNA genome can be single- or double stranded. The RNA genome can be segmented or unsegmented; all well-known to the skilled person.

In an embodiment of the liquid vaccine composition according to the invention, the live virus is a live enveloped virus.

An “enveloped virus” is a well-known type of virus having a phospholipid outer coat. Examples are viruses of: Asfar-, Baculo-, Hepadna-, Herpes-, and Poxvirus families; Corona-, Flavi, and Togavirus families; Arena-, Bunya-, Filo-, Orthomyxo-, Paramyxo-, Pneumo- and Rhabdovirus families; as well as Reo- and Retrovirus families.

The viral envelope is a lipid bilayer surrounding such viruses, which is obtained by the virus upon budding from its eucaryotic host cell. The envelope comprises phospholipids and proteins, and is both very sensitive to damage, as well as critical for the capability of the enveloped virus to infect further host cells. Damage to or degradation of the viral envelope, e.g. resulting from storage for prolonged periods, and/or from storage at elevated temperatures, drastically reduces the capacity to infect of an enveloped virus, i.e. its infectious titre. As for other types of viruses, the stability of a live enveloped virus can effectively be indicated in terms of the effect on its titre i.e. the amount of infectious virus.

In a preferred embodiment of the liquid vaccine composition according to the invention, the live virus is large, has an RNA genome, and is enveloped.

In an embodiment of the liquid vaccine composition according to the invention, the large live enveloped RNA virus is selected from: a Paramyxovirus and a Coronavirus.

As these virus families have members that are relatively large viruses with an envelope and an RNA genome, they are particularly sensitive to biological degradation and loss of titre during storage, especially when in a liquid environment.

For the invention, the virus families indicated herein refer to viruses having the characterising features of their taxonomic group-members such as the morphologic, genomic, and biochemical characteristics, as well as the biological characteristics such as physiologic, immunologic, or pathologic behaviour. The same applies for a reference to a viral genus, or to a name of an individual virus species.

As is known in the field, the classification of a micro-organism in a particular taxonomic group is based on a combination of such features. The invention therefore also includes species of virus from the indicated families or from indicated species names that are sub-classified therefrom in any way, for instance as a subspecies, strain, isolate, genotype, variant, subtype, or subgroup, and the like.

Further, it will be apparent to a person skilled in the field of the invention that while a particular family, subfamily, genus or species of virus for the invention may currently be assigned to that group, however that is a taxonomic classification that could change over time as new insights can lead to reclassification into a new- or different taxonomic group. However, as this does not change the virus itself, or its antigenic repertoire, but only it's scientific name or classification, such re-classified viruses remain within the scope of the invention.

In a preferred embodiment the Paramyxovirus is selected from: a canine para-influenza virus (CPI) and a canine distemper virus (CDV).

In a preferred embodiment the Coronavirus is a bovine coronavirus (BCV) or is an infectious bronchitis virus (IBV).

All these viruses, CPI, CDV, BCV, and IBV, are well known, and attenuated vaccine strains are commonly available for each of them. Alternatively, an attenuated virus can be developed using common procedures.

Samples of these viruses can be obtained from a variety of sources, e.g. as field isolate from a human, or from an animal in the wild or on a farm, or from various laboratories, (depository) institutions, or (veterinary) universities.

The relevance of these viruses in veterinary medicine is described e.g. in well-known handbooks such as: “The Merck veterinary manual” (11^{th} ed., 2016, ISBN-10:9780911910612).

In a preferred embodiment of the liquid vaccine composition according to the invention, the methionine is L-methionine; more preferably the L-methionine is CAS nr. 63-68-3.

In a preferred embodiment of the liquid vaccine composition according to the invention, the ectoine is CAS nr. 96702 Mar. 3.

In a preferred embodiment of the liquid vaccine composition according to the invention, the hydroxyectoine is 5-hydroxyectoine; more preferably the 5-hydroxyectoine is CAS nr. 165542-15-4.

The additive for the compositions of the invention is deliberately added to reach a substantial and effective amount. Therefore it is not a compound present in trace amounts, e.g. resulting from the culturing of the virus or the production of another antigen that is comprised in the liquid vaccine according to the invention. For example, typical culture media for mammalian cells such as e.g. RPMI1640 or DMEM comprise 15-30 mg/L methionine, i.e. 0.0015-0.003% w/w. After culturing, harvesting, and formulation into a vaccine, only minute traces of that methionine will carry-over to a final vaccine product, therefore that does not qualify as presence of an additive for the invention.

Therefore, in an embodiment of the liquid vaccine composition according to the invention, the additive is comprised at 0.05-1% w/w. Preferably the additive is comprised at 0.05-0.5% w/w, or even at 0.1% w/w.

For the invention the additive amount is preferably expressed relative to the weight of the NADES carrier. Also, the indication of a range includes the stated end-points.

In a preferred embodiment of the liquid vaccine composition according to the invention, the additive is methionine.

The compounds for use in the NADES for the invention are readily available in different purities and qualities from a variety of commercial suppliers. Preferably the compounds are used in a pharmaceutical grade quality. Such excipients are for example described in governmental regulations such as the European Pharmacopoeia, the American 9 CFR, and in handbooks such as: The Handbook of Pharmaceutical Excipients (R. Rowe et al., Pharmaceutical press 2012, ISBN 0857110276); Remington: the science and practice of pharmacy (2000, Lippincott, USA, ISBN: 683306472), and: “Veterinary vaccinology” (P. Pastoret et al. ed., 1997, Elsevier, Amsterdam, ISBN 0444819681).

Similarly, the water used for the liquid vaccine composition according to the invention is preferably water of high purity, and is of pharmaceutical quality grade and suitable for parenteral injection; such quality of water is typically sterile, and essentially free from pyrogens, for example: (multiply) distilled water, reverse osmosis water, or water for injection (WFI).

It is well within reach of the skilled person to further optimise a liquid vaccine composition according to the invention. Generally this involves the fine-tuning of the efficacy of the vaccine to further improve the immune-protection it provides. This can be done by adapting the dose, volume, or antigen content of the vaccine, or by application via a different route, method, or regime. All these are within the scope of the invention.

In an embodiment of the liquid vaccine composition according to the invention, the liquid vaccine composition has a water amount of 50% w/w or less, more preferably a water amount of 49, 48, 47, 46, 45, 44, 43, 42, 41, 40, 35, 30, 25, 20 or 15% w/w or less, in that order of preference.

The additive in a liquid vaccine composition according to the invention has a favourable effect in preserving the titre of a live virus comprised in such a composition. This effect is clear already from a water amount of about 5% w/w. Therefore in an embodiment the liquid vaccine composition according to the invention has a minimal water amount of 5% w/w; preferably has a minimal water amount of 7, 10, 12, 15, 20, 30% w/w, in this order of preference.

Combined, the liquid vaccine composition according to the invention thus preferably has a water amount in a range that is a combination of the indicated preferred upper and lower limits, e.g. of between 5-50, 7-45, 10-40% w/w, etc. In a preferred embodiment, the vaccine composition according to the invention has a water amount in the range 20-50% w/w, more preferably 30-50% w/w or even in some cases 35-50% w/w.

As described, the liquid vaccine composition according to the invention can comprise a larger amount of water as compared to NADES-based compositions without an additive, while still preserving live viral titre. The resulting reduction in its viscosity has several benefits for its administration and preparation.

In an embodiment of the liquid vaccine composition according to the invention, the composition has a viscosity that is below 1000 mPa·s, more preferably below 750, 500, 400, 300, 250, 200, 150, or even below 100 mPa·s. Viscosity can be determined by routine methods and by using standard equipment such as described herein.

The liquid vaccine composition according to the invention comprises an amount of live virus that is immunologically effective.

A skilled person in the field of the invention will be more than capable of determining and optimising such an immunologically effective amount of live virus in a liquid vaccine composition according to the invention, e.g. by monitoring the immunological response following vaccination, or (in the case of an animal target) after a challenge infection, e.g. by monitoring the targets' signs of disease, clinical scores, or by re-isolation of the pathogen, and comparing these results to a vaccination-challenge response seen in mock-vaccinated animals.

Methods to determine the amount of live virus in a vaccine dose are well-known in the art, and will typically employ techniques of viral titration such as plaque assays, titration in eggs, in animals, or in micro-titration plates. Consequently such a viral infectivity titre can be expressed e.g. in TCID50, EID50, CID50, or plaque forming units (pfu).

A liquid vaccine composition according to the invention may additionally comprise other compounds, such as an additional antigen or -micro-organism, a cytokine, or an immunostimulatory nucleic acid comprising an unmethylated CpG, etc. Alternatively, the liquid vaccine composition according to the invention, may itself be added to a vaccine.

The liquid vaccine composition according to the invention can advantageously be combined with one or more further antigens, e.g. derived from a micro-organism pathogenic to an intended human- or animal target. Such a further antigen can itself be an infectious micro-organism, or be inactivated, or a subunit. The further antigen can be a biologic- or synthetic molecule such as a protein, a carbohydrate, a lipopolysaccharide, a lipid, or a nucleic acid molecule.

In an embodiment of the liquid vaccine composition according to the invention, the vaccine comprises an additional antigen.

In a preferred embodiment the additional antigen is a bacterial antigen; more preferably the bacterial antigen is one or more selected from Leptospira, Bordetella, Borrelia, Ehrlichia, Mycoplasma, Porphyromonas and Bacteroides.

In a preferred embodiment the additional antigen is an antigen from Leishmania or Microsporum.

The relevance of these pathogens in veterinary medicine is well-known and is e.g. described in the ‘Merck manual’ (supra). Suitable strains of these bacteria are readily available from a variety of sources. The bacterial antigen may be comprised as a live attenuated bacterium, as an inactivated bacterium (a bacterin), or as a part thereof: a lysate, homogenate, extract, fraction, etc.,

In a preferred embodiment, the Leptospira bacteria are one or more selected from:
L. interrogans serogroup Canicola; more preferably serovar Portland-vere or serovar Canicola;
L. interrogans serogroup Icterohaemorrhagiae; more preferably serovar Copenhageni or serovar Icterohaemorrhagiae;
-L. interrogans serogroup Pomona; more preferably serovar Pomona;
-L. interrogans serogroup Australis; more preferably serovar Bratislava or serovar Australis; and
L. kirschneri serogroup Grippotyphosa; more preferably serovar Dadas, serovar Grippotyphosa, or serovar Bananal/Lianguang.

In a preferred embodiment, the Bordetella bacteria are B. bronchiseptica.

In a preferred embodiment of the liquid vaccine composition according to the invention comprising an additional antigen, the additional antigen is a further virus.

In a more preferred embodiment the further virus is one or more selected from: Rabies virus, canine adenovirus type 1 (CAV-1), canine adenovirus type 2 (CAV-2), canine parvovirus (CPV), canine coronavirus (CCV), canine distemper virus (CDV), and canine herpesvirus.

The further virus may be comprised as a live attenuated virus or as an inactivated virus. The relevance of these further viruses in veterinary medicine is well-known and is e.g. described in the ‘Merck manual’ (supra).

In an embodiment of the liquid vaccine composition according to the invention, one or more of the conditions apply, selected from the group consisting of:
the NADES is formed from an organic salt, a polyol, and water; the organic salt is preferably one or more selected from salts of: betaine, proline, carnitine, and choline; the polyol is preferably one or more from a sugar or a sugar-alcohol; the sugar is preferably one or more selected from: fructose, maltose, sucrose, glucose and trehalose; the sugar-alcohol is preferably one or more selected from: glycerol, xylitol, mannitol, and sorbitol;
the NADES is formed from proline, sorbitol and water;
proline is preferably CAS nr. 609-36-9;
sorbitol is D-Sorbitol; preferably D-Sorbitol is CAS nr. 50-70-4;
the proline, sorbitol and water are present in the NADES in molar ratios of proline:sorbitol:water that are between 1:1:1 and 1:1:16; preferably between 1:1:1 and 1:1:15, more preferably between 1:1:1 and 1:1:12, or even between 1:1:1 and 1:1:10;
the live virus is large, i.e. has a diameter of more than 50 nanometres;
the live virus has an RNA genome;
the live virus is a live enveloped virus;
the live virus has both an RNA genome and is enveloped;
the live enveloped RNA virus is selected from: a Paramyxovirus and a Coronavirus;
the Paramyxovirus is selected from: a canine para-influenza virus (CPI) and a canine distemper virus (CDV);
the Coronavirus is a bovine coronavirus (BCV) or is an infectious bronchitis virus (IBV);
the ectoine is preferably CAS nr. 96702-03-3;
the hydroxyectoine is 5-hydroxyectoine; preferably the 5-hydroxyectoine is CAS nr. 165542-15-4;
the additive is comprised at 0.05-1% w/w; preferably the additive is comprised at 0.05-0.5% w/w, or even at 0.1% w/w;
the additive is methionine;
the methionine is L-methionine; preferably the L-methionine is CAS nr. 63-68-3;
the liquid vaccine composition has a water amount of 50% w/w or less; more preferably a water amount of 49, 48, 47, 46, 45, 44, 43, 42, 41, 40, 35, 30, 25, 20 or 15% w/w or less, in that order of preference;
the liquid vaccine composition has a minimal water amount of 5% w/w; preferably has a minimal water amount of 7, 10, 12, 15, 20% w/w, in this order of preference;
the liquid vaccine composition has a viscosity that is below 1000 mPa·s, more preferably below 750, 500, 400, 300, 250, 200, 150, or even below 100 mPa·s;
the liquid vaccine composition comprises an additional antigen; preferably the additional antigen is a bacterial antigen; more preferably the bacterial antigen is one or more selected from Leptospira bacteria and from Bordetella bacteria;
the Leptospira bacteria are one or more selected from:
   L. interrogans serogroup Canicola; more preferably serovar Portland-vere or serovar Canicola;
   L. interrogans serogroup Icterohaemorrhagiae; more preferably serovar Copenhageni;
   L. interrogans serogroup Pomona; more preferably serovar Pomona;
   L. interrogans serogroup Australis; more preferably serovar Bratislava; and
   L. kirschneri serogroup Grippotyphosa; more preferably serovar Dadas, serovar Grippotyphosa, or serovar Bananal/Lianguang;
the Bordetella bacteria are B. bronchiseptica;
for the liquid vaccine composition comprising an additional antigen, the additional antigen is a further virus; and
the further virus is one or more selected from: Rabies virus, canine adenovirus (CAV), canine parvovirus (CPV), and canine coronavirus (CCV).

In an embodiment of the liquid vaccine composition according to the invention, the NADES is formed from proline, sorbitol and water, which are present in the NADES in molar ratios of proline:sorbitol:water that are between 1:1:1 and 1:1:10; the additive is comprised at 0.1% w/w; the additive is methionine; the live virus is one or both selected from CPI and CDV; the liquid vaccine composition comprises an additional antigen being one or more bacterial antigen selected from Leptospira bacteria and Bordetella bacteria; and the liquid vaccine composition comprises a further additional antigen being one or more further virus selected from: canine adenovirus (CAV), canine parvovirus (CPV), and canine coronavirus (CCV).

The liquid vaccine composition according to the invention can be produced using common techniques and materials. Details and examples of such a method, a use, or a process for preparing a liquid vaccine composition according to the invention, are described herein, and such procedures are readily applicable by a person skilled in the art, using routine materials and methods.

For example, the NADES for the invention can be produced at industrial scale; being less viscous makes this part of the manufacture more convenient now. An additive as defined for the present invention is then added; this may be added at different stages, e.g. during the formation of the NADES, to the NADES after it has formed, to the live virus preparation before that is mixed with the NADES, or to a mixture of NADES+virus.

The mixture is then filled-out into appropriately sized containers. The various stages of the manufacturing process will be monitored by adequate tests, for instance by immunological tests for the quality and quantity of the virus; by microbiological tests for sterility, and absence of extraneous agents; and ultimately by vaccination-studies in animals for confirming efficacy and safety. After completion of the testing for quality, quantity and sterility, the vaccine product can be released for sale.

All these are well known to a skilled person, and general techniques and considerations that apply to the preparation of vaccines under well-known standards for pharmaceutical production are described for instance in governmental directives and regulations (Pharmacopoeia, 9CFR, supra) and in well-known handbooks (Pastoret, Remington, supra).

Therefore in a further aspect the invention relates to a method for preparing the liquid vaccine composition according to the invention, the method comprising the steps of:
preparing the NADES as defined for the invention,
admixing said NADES with the additive as defined for the invention, and
admixing the NADES and additive with a composition comprising the live virus.

Conveniently these steps of the method according to the invention can be performed separated in time and/or in place, with intervening storage, transport, or even further steps. This will allow flexibility in planning and logistics of operation.

The preparation of the NADES for use in the method according to the invention can be done using routine tools and methods. One convenient way is to apply heating to the combined compounds to a temperature that facilitates them to mix and solubilise, but that does not damage them, e.g. up to about 80° C. The heating can e.g. be done in combination with stirring or with sonification.

Also, one of the components of the NADES can initially be dissolved in water in more or less concentrated form, after which it can be mixed with (a) further component(s) to form the NADES for the invention by evaporating off the surplus of water.

The additive as defined for the invention can be mixed with the NADES after that has formed, or can be added to the compounds of the NADES before that is formed. The additive can be added as a dry salt; this helps to control the total amount of water in the liquid vaccine according to the invention. Alternatively the additive can be added from an aqueous stock solution. Evidently the amount of water added to the composition in this way needs to be factored in when making of the liquid vaccine composition according to the invention.

The composition comprising the live virus for use in the method according to the invention can conveniently be derived in different ways, advantageously from an in vitro culture of the virus on a suitable cell-line. Virus-cell cultures can be made at any desired size or volume, up to several 1000 litres in large scale industrial size fermenters applying automated controls and monitoring techniques.

The virus is then harvested from the culture in a suitable way, e.g. as the supernatant, the cell-pellet, or as a whole culture; this can be fine-tuned to the properties of the virus and of the culture system used. The virus preparation to be used in the method according to the invention can be treated further when desired, using further process steps such as concentration, e.g. by ultra-filtration. The virus composition is then checked for quality and quantity. All this is well-known to a skilled person.

The admixing of the NADES components, the additive, and the virus in the method according to the invention, is preferably done using aseptic techniques.

The order and the way of admixing is not critical, in that the NADES may be added to the virus composition, or the other way around, and the additive can be added at any stage during this process. Preferably, any mechanical mixing used will be low speed and low impact, to be gentle on the live virus. Further, the mixing and/or filling can be done in a protective atmosphere, e.g. using Nitrogen or Argon gas, to reduce oxygen exposure.

Convenient equipment for mixing and filling the liquid vaccine composition is available from a variety of suppliers of process-equipment.

In an embodiment of the method for preparing the liquid vaccine composition according to the invention, the steps of preparing the NADES and adding the additive are combined, whereby the NADES ingredients and the additive are combined, after which the NADES is formed as described. For example: proline, sorbitol, and methionine are combined as dry salts, some water is added, and the NADES+additive is formed during heating, optionally with sonication.

In a further aspect the invention relates to the use of an additive as defined for the invention for stabilising a live virus in a liquid vaccine composition according to the invention.

In an embodiment of the use of an additive according to the invention, the additive is comprised in the liquid vaccine composition at 0.05-1% w/w. Preferably the additive is comprised at 0.05-0.5% w/w, or even at 0.1% w/w.

For the invention, “stabilising” refers to the reduction of the loss of virus titre over time, as defined above.

In a further aspect the invention relates to a method for stabilising a live virus in a liquid vaccine composition according to the invention, the method comprising the step of comprising an additive as defined for the invention in said liquid vaccine composition.

In an embodiment of the method for stabilising according to the invention, the additive is comprised in the liquid vaccine composition at 0.05-1% w/w. Preferably the additive is comprised at 0.05-0.5% w/w, or even at 0.1% w/w.

As described, the present invention enables liquid vaccine compositions of live viruses, which composition has less viscosity, but retains good viral stabilisation. The compositions can therefore advantageously be used as a vaccine for a variety of targets and diseases.

Therefore in a further aspect the invention regards the liquid vaccine composition according to the invention for use in a method for protecting a human or animal target against infection and/or disease caused by a pathogenic form of the virus comprised in said liquid vaccine composition.

The “method for protecting” comprises a step of administering the liquid vaccine composition according to the invention to a target in need of such protection. As described, this administration can be done in a variety of ways, in particular because the NADES-based vaccine now has a reduced viscosity.

Therefore, in an embodiment the use in a method for protecting according to the invention comprises the administration by mucosal route; preferably the mucosal route involves a route selected from an ocular, nasal and oral route. The mucosal route can also be a combination, e.g. such as oculo-nasal, or nasal-oral.

Evidently, the method for protecting according to the invention may also be done by administration by parenteral route, i.e. through the skin, e.g. by intramuscular, intraperitoneal, intradermal, submucosal, or subcutaneous route. Preferred route of parenteral administration of the liquid vaccine composition according to the invention is by intramuscular- or by subcutaneous injection.

Further the method for protecting according to the invention can involve administering the liquid vaccine composition according to the invention by enteral route.

Alternatively, the liquid vaccine can be administered via a method of mass administration, e.g. via drinking water, coarse spray, atomisation, on-feed, etcetera.

The method for protecting according to the invention can be applied either as a prophylactic- or as a therapeutic treatment, or both.

The liquid vaccine composition according to the invention can serve as an effective priming vaccination, which can later be followed and amplified by a booster vaccination, with the same or with a different vaccine.

The targets for the liquid vaccine composition for use according to the invention are humans or animals in need of a vaccination against infection and/or disease caused by a pathogenic form of a live virus that is comprised in the vaccine. The age, weight, sex, immunological status, and other parameters of the target to be vaccinated are not critical, although it is favourable to vaccinate healthy, uninfected targets, and to vaccinate as early as possible to prevent any field infection.

In an embodiment of the method for protecting according to the invention, the target is a mammalian or an avian species; preferably the mammalian species is a bovine or a canine.

In embodiments of the liquid vaccine composition for use according to the invention the features of the liquid vaccine and the live virus are all as disclosed herein for the invention.

In a further aspect the invention regards a method for vaccinating a human or animal target, the method comprising the administration of a liquid vaccine composition according to the invention to said target.

In embodiments of the method for vaccinating according to the invention, the features of the administration and the liquid vaccine are all as disclosed herein for the invention.

The invention is described herein in various aspects and embodiments. It should be understood that any combination of these are considered to be within the scope of the invention. However merely for conciseness, not every possible combination is outlined herein in full.

The invention will now be further described by the following, non-limiting, examples.

### EXAMPLES

### Example 1: Comparison of the Properties of Different Compositions

In the field of live virus vaccines, most compositions described are freeze-dried cakes. Some publications describe liquid preparations, which are typically aqueous compositions, i.e. based on water as the carrier, not on a NADES. These aqueous compositions of live virus differ from NADES-based liquid vaccines mainly in their DSC profile, and in the amount-, the concentration-, and the availability of the water that is present. As some of those aqueous compositions may superficially resemble the compositions of the invention, they are analysed and compared here in more detail, which illustrates clearly that they are in fact essentially different.

Aqueous Compositions from Literature

WO 2014/029702 and related publications such as: WO 2014/140239, WO 2015/121463 and US 2014/0271710, describe a number of liquid-stable preparations of a variety of viruses, in compositions with a sugar-alcohol and one or more amino acids. One exemplary composition, taken from '702, comprises: 30% w/w Sorbitol and 0.6 M Arginine in water. Analysing this composition in more detail shows the following. Incorporating the (estimated) density of the composition at 1.1 g/ml, means 1100 grams are present\litre. As Sorbitol is at 300 g/g, that makes 330 g/l, and having a Mw of 182 g/mole, Sorbitol is thus at 1.8 M. Similarly, Arginine is at 0.6 M, and by having a Mw of 174.2 g/mole, this corresponds to 105 g/l Arginine. This leaves the water at 1100−330−105=665 grams/litre, and at 18 g/mole, this is thus 37 M of water. This corresponds to an amount of water of 60.5% w/w. Also when this composition was measured for its water activity, its Aw=0.89.

In conclusion: the liquid compositions described in WO 2014/029702 and related publications, have a water content of 60%, a water concentration of 37 M, and a water activity of 0.89. All these are very much above the limits and preferred values for the NADES-based compositions of the invention.

### NADES Properties

When comparing the above aqueous compositions to the NADES-based compositions of the invention, it is immediately apparent that those have essentially different compositions and properties. For example:
A NADES of proline:sorbitol:water of 1:1:2.5 molar ratios, has a water activity of Aw=0.39, and has a water amount of only 13.2% w/w,
Even a NADES with some more water having proline:sorbitol:water at 1:1:10 molar ratios, has an Aw=0.76, and has a water amount not more than 37.7% w/w.

See Table 1 for more details. That table also outlines the glass-transition temperatures (Tg) for the two extreme NADES shown, and the reduction in viscosity obtained when preparing a NADES with more water.

The Tg of a sample was determined by differential scanning calorimetry (DSC) on a Q2000 DSC apparatus from TA Instruments using Thermal Analysis software. For a full DSC profile, samples were first equilibrated at 40° C. and then cooled to −90° C. at 5° C./min, followed by equilibration for 5 min at −90° C. after which samples were heated again to 40° C. at 5° C./min. This cycle of cooling and heating was repeated a second time on the same sample to investigate phase hysteresis. The Tg of a sample was determined by the software, based on the heating curve at the midpoint of the stepwise shift in the baseline, indicating a change in thermal conductivity from the increased mobility during the glass transition.

Viscosity was determined using standard conditions: at 20° C., using a Brookfield™ DV-|+viscometer, utilising spindle type No. 62, for 30 seconds at 60 r.p.m., in a standard measuring cup of 80 ml volume.

TABLE 1
Properties of NADES with increasing amounts of water

| | | | amount of | water | water | | |
|---|---|---|---|---|---|---|---|
| molar ratio | | | water | activity | concen- | Tg | viscosity |
| P:S:W | | | (% w/w) | (a_{w}) | tration (M) | (° C.) | (mPa · s) |
| 1 | 1 | 2.5 | 13.2 | 0.39 | 9.7 | −41.9 | 5197 |
| 1 | 1 | 5.0 | 23.3 | 0.61 | 16.6 | −63.6 | 289 |
| 1 | 1 | 7.5 | 31.3 | 0.66 | 21.8 | −73.3 | 75 |
| 1 | 1 | 10 | 37.7 | 0.76 | 25.7 | −82.2 | 33 |

### NADES-Based Compositions According to the Invention

For the preparation of a liquid vaccine composition according to the invention, several volume parts of a NADES are mixed with a volume part of an aqueous preparation of live virus. Typically 9 volume parts of NADES are mixed with 1 volume part of virus suspension, but in the spirit of the invention, lower relative amounts of the NADES can also be used, or the other way around: higher relative amounts of suspension of virus (and other antigens) can be added, which leads to a further dilution of the NADES. This reduces viscosity but also may affect virus stability, which in turn can be prevented to a large extend by the inclusion of an additive as defined for the invention.

The admixing of the antigen suspension raises the water amount in the final liquid vaccine composition slightly, although that is not a linear effect: at higher levels of initial water amount in the NADES, the relative increase in water amount from such a dilution with virus sample, is less than at lower starting water amounts. This was analysed e.g. by adding 1 part of pure water, to 9 parts of a NADES composed of proline:sorbitol:water (P:S:W) at 1:1:2.5 molar ratios. This resulted in an increase of the amount of water from 13.2 for the NADES to 25.8% w/w for the NADES-based composition. For other P:S:W NADES mixed at 9+1 with water, the water amount increases were as follows: the 1:1:5 went from 23.3 to 37.0% w/w; and the 1:1:10 went from 37.7 to 50% w/w. In this last case, the Aw increased from 0.76 to 0.80.

### Example 2: Different Additive Compounds Tested

A number of compounds were tested for their potential to compensate the negative effect on viral stability from the increased amount of water in the liquid vaccine compositions according to the invention. Different virus types were used, and different incubation temperatures, to get the broadest impression of additive efficacy in the context of a NADES-based virus composition.

### 2.1 Canine Parainfluenza Virus

CPI is a Paramyxovirus, and it was admixed into a NADES carrier that contained water and different additives, and incubated for increasing time and at different temperatures. In short:

The NADES used was proline:sorbitol:water (P:S:W) at 1:1:10 molar ratio. The ingredients of the NADES and 0.1% w/w of one of different additives was mixed and sonified in a heated water bath for solubilisation. The same NADES but without additive was used as control.

After cooling, 0.9 ml of NADES+additive, or control NADES, was filled out into a number of 3 ml glass vials. At the start of the stability experiment, 0.1 ml of a solution of CPI at 10\hat{ }9 TCID50/ml was added to each vial; the vial was closed, and mixed thoroughly. Water amount was 38% w/w before, and 50% w/w after admixing the additive and the virus.

The vials for t=0 were titrated immediately on Vero cells; standard 96 well plates had been prepared the day before with Vero cells at 1×10\hat{ }5 cells/ml in 100 μl standard culturing medium, and these were incubated overnight at 37° C., under 5% CO₂.

At each time-point to be tested, a vial of the virus+NADES composition was taken up into 1 ml culturing medium, and stepwise 1:10 dilutions were prepared by taking 0.2 ml and adding to 1.8 ml medium, for 7 consecutive cycles more. 100 μl of each dilution was then added to each well of a 96 well plate with Vero cells, and incubated for 7 days. Next, cpe was read, and titre was determined as a TCID50, expressed in Log 10/ml.

Except for the t=0 samples, the other samples were stored at room temperature (i.e. 20° C.) for at least 24 weeks. At the appropriate time points: 4, 8 and 24 weeks after start of the experiment, a vial was taken and its content was titrated as described.

The different additives tested here were: citric acid monohydrate, ectoine, EDTA, methionine, and lecithin, all used at 0.1% w/w relative to the weight of the NADES. A further control series was included, whereby the CPI was put into phosphate buffered saline (PBS), and treated and stored the same way.

Results for the incubation of CPI virus in the different compositions are presented in the graph of FIG. 3. As is quite clear, CPI is a rather stable virus, which was detectable in the PBS composition even after incubation for 8 weeks at room temperature. However the PBS sample was negative for live CPI at 24 weeks. In the control NADES composition, even at 24 weeks still a titre of 5.9 of CPI was detectable. The effect of the different additives was then compared: citric acid, EDTA, and lecithin did not provide a clear improvement to the stabilisation by the control NADES after 24 weeks. However, the samples with ectoine or methionine kept their titres much better over time.

The fact that not all samples at t=0 scored a titre of 10\hat{ }8 is partly test variability, but also an effect of the initial in-mixing of the virus and the NADES, which can sometimes cause a small drop in titre of itself.

It is clear that even a NADES-based liquid composition according to the invention cannot stabilise CPI at its starting level for extended time at elevated temperature. Nevertheless, it was impressive to learn that CPI virus could be stored at room temperature for at least half a year in liquid conditions, and only suffered a limited loss of about 1.5 Log 10/ml in titre. This illustrates there is no need for freeze-drying, and that a NADES-based composition with a substantial amount of water can still stabilise live virus effectively.

### 2.2 Bovine Coronavirus

BCV was used for a similar series of experiments with different additives. BCV was found to be more sensitive then CPI to prolonged storage in liquid conditions, as it showed strong loss of titre, even at 4° C.

The experimental set-up was the same as described for CPI: the BCV was used at a titre of 6.5 Log 10 TCID50 in each vial. Combination with NADES was at 1+9 v/v, and control NADES was the P:S:W 1:1:10 composition, incubation was for up to 2 weeks at 28° C., or for up to 13 months at 4° C. BCV was titrated on Madin Darby Bovine Kidney cells in microtitration plates. Infection (live virus replication) was detected by IFT. Additives were used at 0.1% w/w and were mixed-in during the preparation of the NADES.

Results for the BCV incubations are presented in FIG. 4, with the incubations at 28 and at 4° C. in panels A and B respectively.

The incubation at 28° C. served as an expedited stability test. In these conditions, the control NADES was not able to stabilise the BCV at a relevant level: after one week 2.5 Log 10 of viral titre was lost, and after two weeks no BCV was detectable anymore. This was not improved by the use of citrate or vitamin C as additive, on the contrary, these even showed increased loss of titre. However, and surprisingly, hydroxyectoine and methionine were able to maintain BCV titre to around 3 Log 10 after incubation for 2 weeks at 28° C. in liquid conditions; that is an impressive performance by any standard.

More focussed on practical conditions, were the incubations at 4° C., and these were extended for a very long period. Among the compositions tested a further control was a storage in pure water. This showed complete loss of BCV titre at the 2 months time point. The control NADES showed a gradual loss of titre after the first month, until at 13 months no live BCV was detectable anymore. Slightly less were the results from the use of citrate or ferro-citrate as additives, but vitamin C was much worse.

However, and again: hydroxyectoine and methionine were able to add an impressive improvement to the stabilising capacity of the NADES control, even after storage in liquid conditions for 13 months at 4° C.

### Example 3: Further Viruses Tested

To expand on these favourable results, more viruses were tested:

### 3.1 Infectious Bronchitis Virus

IBV is an avian Coronavirus, and it was used in tests with different NADES compositions, by incubation at 4° C. up to 36 weeks.

The experimental set-up was essentially as described above: IBV strain MA5 was mixed 1+9 v/v with a NADES that was a P:S:W at 1:1:5 ratio with 0.1% w/w methionine. As control IBV was mixed in PBS. The IBV samples were titrated on fertilized eggs; after incubation for 3 days, allantoic fluid was collected, and this was used in antigenic mass Elisa to quantify IBV.

Results of the incubations of IBV are presented in FIG. 5.

Evidently, in PBS there was a rapid loss of IBV titre, and no live virus was detected anymore after 36 weeks at 4° C. Remarkably however, the 1:1:5 NADES with methionine composition was able to retain most of the live IB virus, even after incubation for 36 weeks at 4° C. in liquid conditions.

### 3.2 Canine Distemper Virus

CDV is a further Paramyxovirus, and it was used for stability tests at room temperature up to two months, and at 4° C. up to 8 months or even up to 23 months. CDV strain Onderstepoort was used at 6.1 Log 10 TCID50 starting titre. The CDV virus had been grown, and was titrated on Vero cells. The NADES used was P:S:W 1:1:5, with or without 0.1% w/w methionine. In the first series of samples (section 3.2.1 below) CDV was taken up into the NADES at 1+9 v/v, which increased the water content from 23.3 to 26.2. % w/w. In a parallel series of samples (section 3.2.2 below) a CDV virus sample was used which included further viruses: canine adenovirus, canine parvovirus and canine parainfluenza virus. This larger virus sample increased the water content of the P:S:W 1:1:5 NADES from its initial 23.3 to a final 40.1% w/w.

As a further control, a CDV sample was taken up into a classic liquid stabiliser composition called ‘IDK’ which contained NZ amine and sorbitol in an aqueous phosphate buffer. The IDK stabiliser is a typical aqueous composition and has a water content of 87.4% w/w.

Results for the CDV incubations are presented in FIG. 6, with the incubations in a composition with 27% w/w water at 20° C. and at 4° C. in panels A and B respectively, and the results for the incubation in a composition with 40% w/w water in panel 6 C.

3.2.1: CDV Stability in a NADES-Based Composition with 26% w/w Water

At 20° C., the control NADES did slow the drop in virus titre somewhat but not effectively. The classic aqueous stabiliser IDK was even much worse in that at the 2 month time point no live CDV was detectable anymore. However, the NADES+methionine was able to improve upon the stability seen with the control NADES.

At 4° C. the same trend emerged, but the effect was more pronounced: the IDK stabiliser was able to maintain some CDV titre up to 8 months of incubation at 4° C. in liquid conditions. However the NADES with methionine provided excellent stabilisation, which was also much better than that from the control NADES.

3.2.2: CDV Stability in a NADES-Based Composition with 40% w/w Water

In FIG. 6 C, the aqueous stabiliser was IDK, same as indicated above.

The samples having 40% w/w water were incubated at 4° C. The CDV in the NADES control gradually lost its titre, and no live CDV was detectable anymore after 23 months incubation at 4° C. in liquid conditions. However by the addition of 0.1% w/w methionine, this loss of titre could be delayed by almost one further year!

### LEGEND TO THE FIGURES

FIG. 1

Figure from literature, reproduced herein. The original is FIG. 1 of WO 2020/201048, showing the differential scanning calorimetry (DSC) glass-transition profile of an intact NADES. Specifically this is the DSC profile of a NADES with proline, sorbitol, and water at 1:1:2.5 molar ratios.

FIG. 2

Figure from literature, reproduced herein. The original is FIG. 4 of Qiao et al., (2018, Appl. Microbiol. Biotechnol., vol. 102, p. 5695-5705), showing DSC profiles with the melt- and crystallisation enthalpy of over-diluted NADES which became aqueous compositions.

FIG. 3

Results for the incubation of CPI in compositions with different additives. Incubations were done at 20° C. Details are described in Example 2.

FIG. 4

Results for the incubation of BCV in compositions with different additives. Incubations were done at 28° C. (FIG. 4A) or at 4° C. (FIG. 4 B). Details are described in Example 2.

FIG. 5

Results for the incubation of IBV in a NADES-based composition comprising methionine as additive. Incubations were done at 4° C. Details are described in Example 3.

FIG. 6

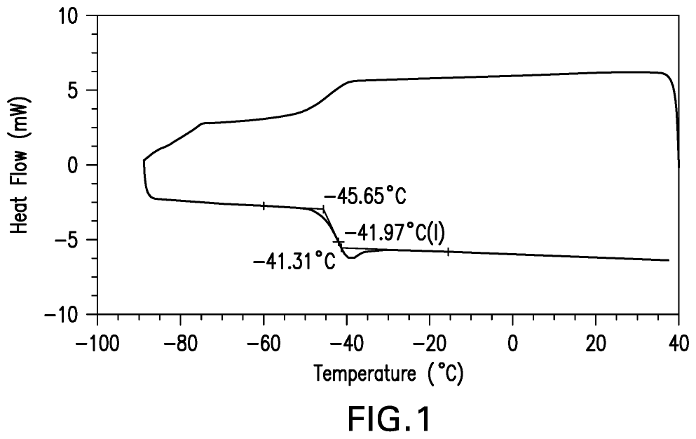

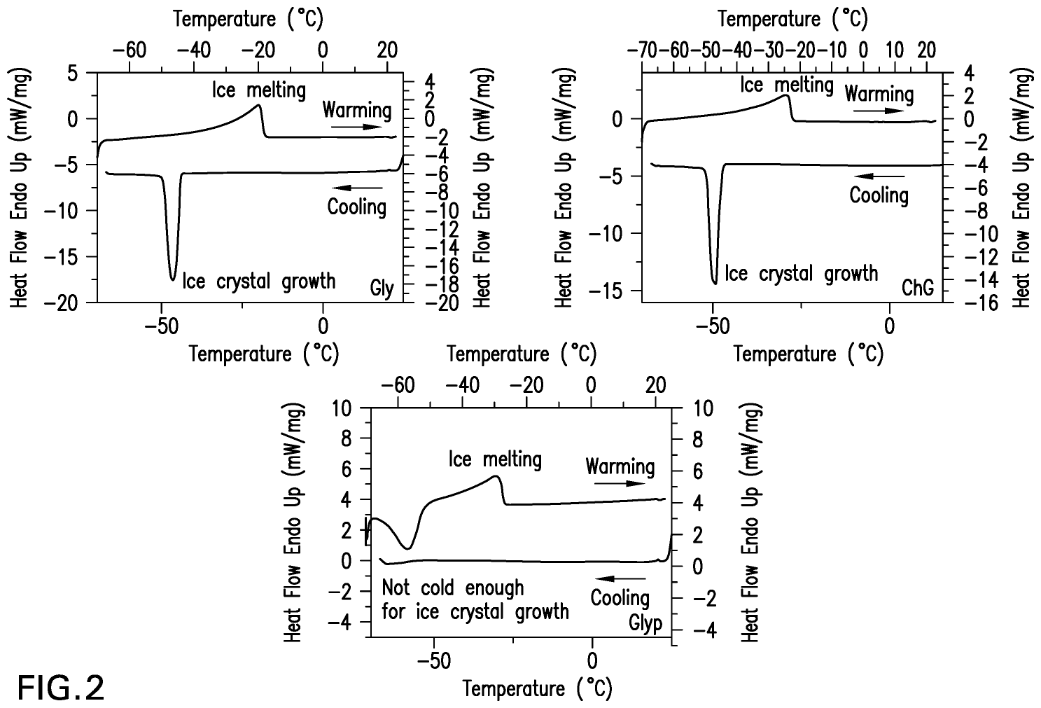

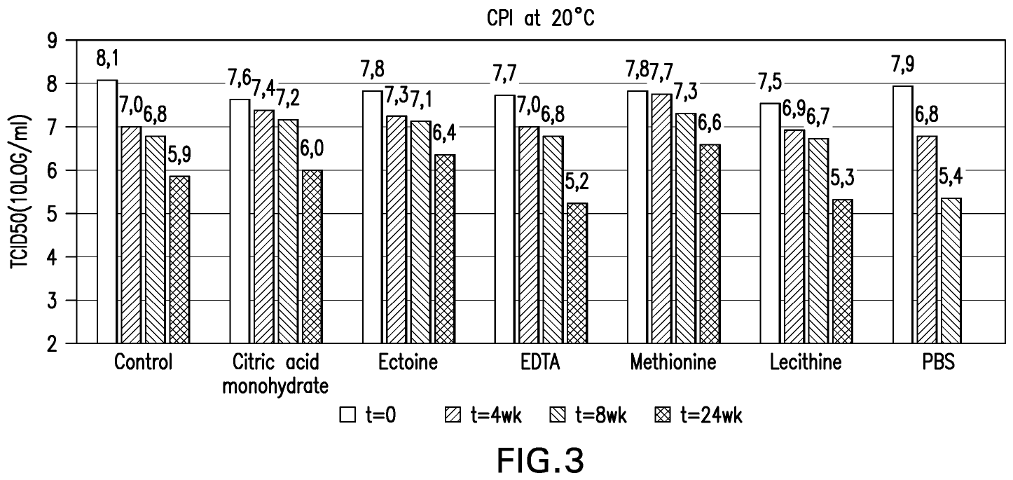

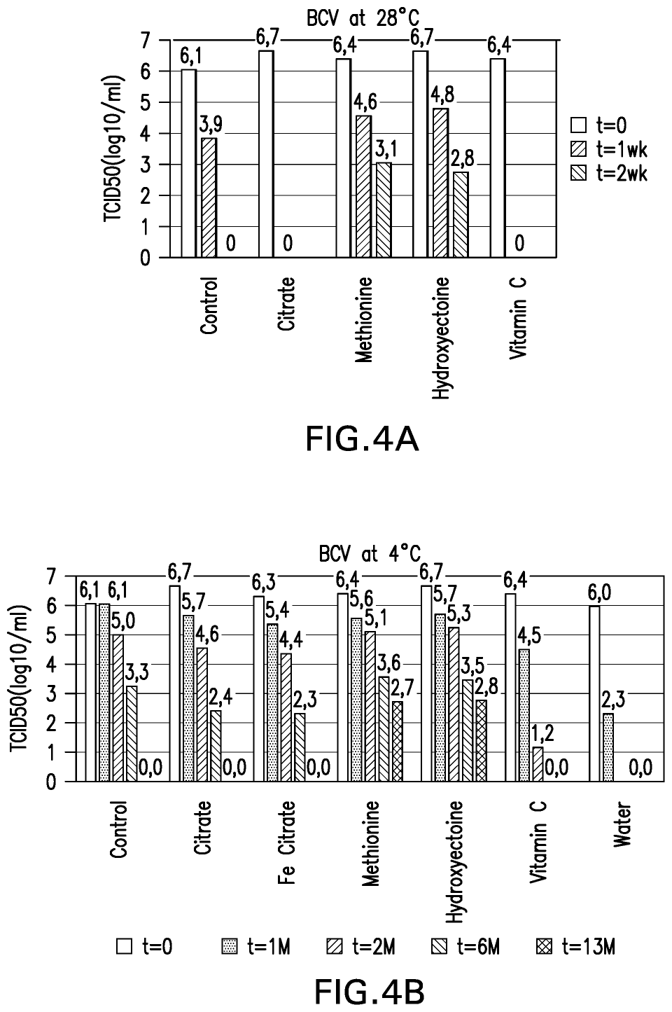

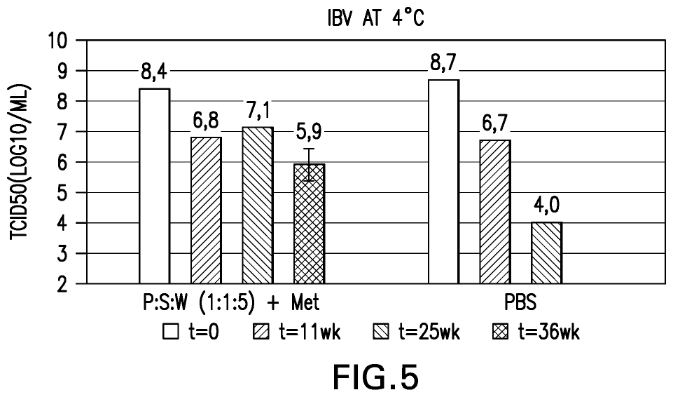

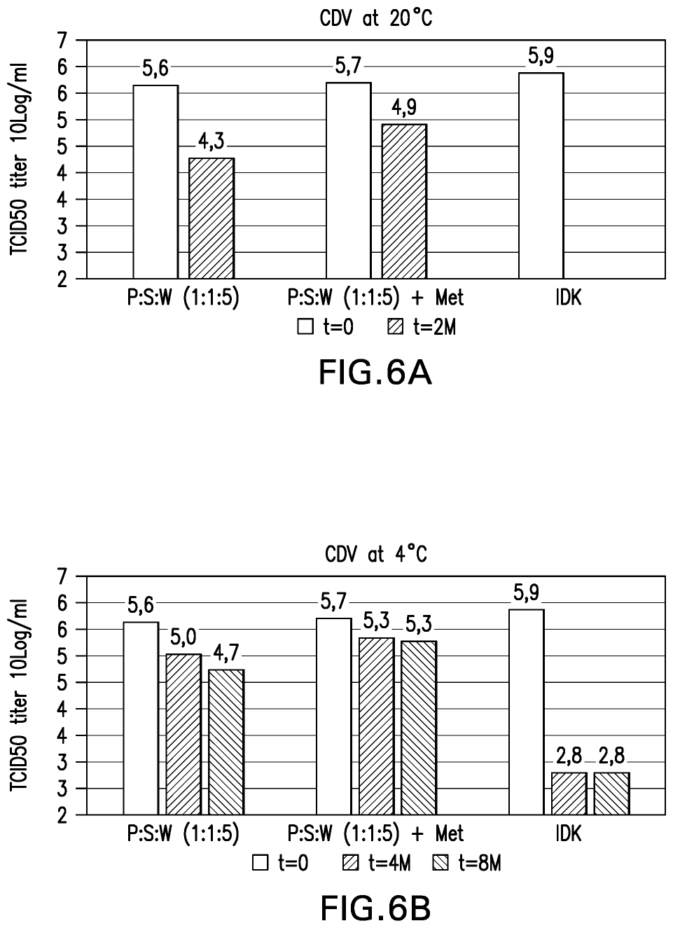

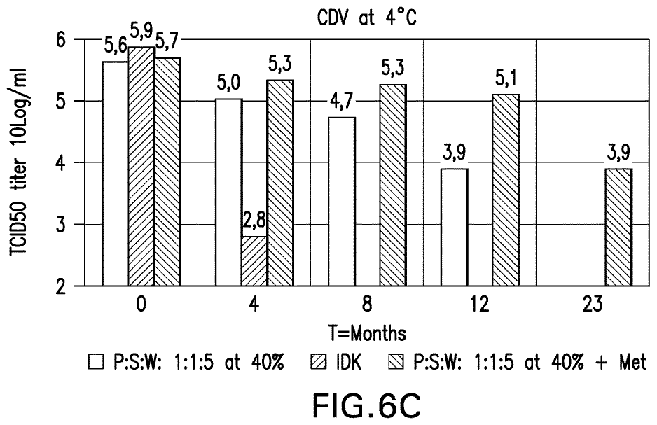

Results for the incubation of CDV in NADES-based liquid compositions with different amounts of water. Stability tests were done in a composition with 27% w/w water at 20° C. (FIG. 6A) or at 4° C. (FIG. 6 B); alternatively incubations were done in a composition with 40% w/w water at 4° C. (FIG. 6 C). Details are described in Example 3.

1. A liquid vaccine composition comprising a live virus and a pharmaceutically acceptable carrier, the carrier being a natural deep-eutectic solvent (NADES), and the vaccine having a water activity of at most 0.8, characterised in that the liquid vaccine composition has a water amount of up to 50% w/w, and the vaccine also comprises an additive selected from methionine, ectoine, and hydroxyectoine.

2. The liquid vaccine composition according to claim 1, characterised in that the NADES is formed from proline, sorbitol and water.

3. The liquid vaccine composition according to claim 2, characterised in that the proline, sorbitol and water are present in the NADES in molar ratios of proline:sorbitol:water that are between 1:1:1 and 1:1:10.

4. The liquid vaccine composition according to any one of claims 1-3, characterised in that the additive is comprised at 0.05-0.5% w/w.

5. The liquid vaccine composition according to anyone of claims 1-4, characterised in that the live virus is a live enveloped virus.

6. The liquid vaccine composition according to claim 5, characterised in that the live enveloped virus is selected from: a Paramyxovirus and a Coronavirus.

7. The liquid vaccine composition according to claim 6, characterised in that the Paramyxovirus is selected from: a canine para-influenza virus (CPI) and a canine distemper virus (CDV).

8. A method for preparing the liquid vaccine composition according to any one of claims 1-7, the method comprising the steps of:
preparing the NADES as defined in any one of claims 1-4,
admixing said NADES with the additive as defined in one of claim 1 or 4, and
admixing the NADES and additive with a composition comprising the live virus.

9. Use of an additive selected from methionine, ectoine, and hydroxyectoine, for stabilising a live virus in a liquid vaccine composition as defined in any one of claims 1-7.

10. The liquid vaccine composition according to any one of claims 1-7, for use in a method for protecting a human or animal target against infection and/or disease caused by a pathogenic form of the virus comprised in said liquid vaccine composition.

11. A method for vaccinating a human or animal target, the method comprising the administration of a liquid vaccine composition according to any one of claims 1-7 to said target.
